(19) United States
(12) Patent Application Publication
(10) Pub. No.: US 2026/0306747 A1
(43) Pub. Date: Oct. 8, 2026

(54) MEDICAMENT DELIVERY SYSTEM WITH DOSE DIVIDER

(71) Applicant: Sanofi Pasteur, Inc., Swiftwater, PA (US)

(72) Inventor: Stephan Riedel, Frankfurt (DE)

(21) Appl. No.: 19/481,804

(22) PCT Filed: May 10, 2024
(86) PCT No.: PCT/US2024/028771

§ 371 Date: Nov. 5, 2025

(30) Foreign Application Priority Data
May 11, 2023 (EP) 23172948.4

(51) Int. Cl. A61M 5/315; A61M 11/00

(52) U.S. Cl. CPC A61M 5/31501; A61M 5/31536; A61M 5/31571; A61M 5/3158; A61M 5/31595; A61M 11/007; A61M 2005/31508; A61M 2202/0007; A61M 2202/04; A61M 2205/276; A61M 2205/586

(57) ABSTRACT

A medicament delivery system comprises a syringe and a plunger rod. The plunger rod comprises a first region of a first outer configuration and a second region of a second outer configuration that is different to the first outer configuration. A blocking element is configured to be provided at a proximal end of the syringe and disposed around the plunger rod, and which permits the plunger rod to move over a first range of motion relative to the syringe to expel a first dose of medicament, and blocks the plunger rod from moving over a subsequent range of motion relative to the syringe to expel further medicament. The blocking element is configured to be removable from the plunger rod. The plunger rod is free to move over the subsequent range of motion relative to the syringe to expel further medicament once the blocking element is removed from the plunger rod.

### TECHNICAL FIELD

The present invention relates to a medicament delivery system configured to facilitate delivery of a medicament in separate doses.

### BACKGROUND

Medicament delivery devices may typically comprise a container of medicament having an outlet through which the medicament can be expelled from the container during medicament delivery, and a plunger rod operable by a user to cause the delivery of medicament from the outlet. Such plunger rod may, for example, comprise a plunger or piston within the medicament container which can be moved into the medicament container to dispense medicament.

Some medicaments are intended to be delivered in two or more doses from a single container. In such cases, a user may be required to manipulate the plunger rod over a first range of motion to dispense a first dose, and then manipulate the plunger rod over one or more subsequent range(s) of motion to dispense subsequent doses. Some patients may have difficulty in manipulating the plunger rod to accurately dispense the intended separate doses with the intended quantities of medicament in each dose. Alternatively, patients may accidentally dispense unintended quantities of medicament, or dispense all of the medicament at once, through inexperience with the device or misunderstanding of device usage instructions.

A known device for delivering divided doses of medicament is available as FluMist™ device. Other known devices for delivering divided doses of medicament are disclosed in WO 2022/049080 and WO 2022/049086.

Respiratory syncytial virus (RSV) is an important cause of severe acute lower respiratory illness (LRI) in infants and children and a common cause of severe pneumonia requiring hospital admission in children worldwide. According to global estimates, RSV caused approximately 33 million cases of LRI and approximately 118,000 deaths in children <5 years of age in 2015. Greater than 80% of all RSV-associated LRIs (RSV-LRIs) and more than 50% of the RSV-associated deaths in low-and middle-income countries were estimated to occur in infants≥6 months old.

### SUMMARY

According to the present disclosure, there is provided a medicament delivery system comprising a syringe comprising a chamber for containing medicament and an outlet at a distal end of the syringe through which medicament can be expelled from the chamber, a plunger rod mounted within the syringe and slidable relative to the syringe to cause medicament to be expelled from the chamber when medicament is within the chamber, wherein the plunger rod comprises a first region of a first outer configuration and a second region of a second outer configuration that is different to the first outer configuration, and a blocking element configured to be arranged at a proximal end of the syringe and disposed around the plunger rod, and which permits the plunger rod to move over a first range of motion relative to the syringe to expel a first dose of medicament, and blocks the plunger rod from moving over a subsequent range of motion relative to the syringe to expel further medicament, wherein the blocking element is configured to be removable from the plunger rod, and wherein the plunger rod is free to be moved over the subsequent range of motion relative to the syringe to expel further medicament once the blocking element is removed from the plunger rod.

Optionally, when the plunger rod is located along the first range of motion relative to the syringe, the blocking element permits the plunger rod to move over the first range of motion, and the blocking element becomes removable from the plunger rod once the plunger rod has completed the first range of motion.

Optionally, when the plunger rod is located along the first range of motion relative to the syringe, the blocking element permits the plunger rod to move over the first range of motion and the blocking element is not removable from the plunger rod; and the blocking element is removable from the plunger rod once the plunger rod has completed the first range of motion.

Optionally, when the plunger rod is located along the first range of motion relative to the syringe, the blocking element is not removable from the plunger rod; and when the plunger rod is located along the subsequent range of motion relative to the syringe, the blocking element is removable from the plunger rod.

Optionally, when the plunger rod is located along the first range of motion relative to the syringe, the blocking element is not removable from the plunger rod.

Optionally, when the plunger rod is located along the subsequent range of motion relative to the syringe, the blocking element is not removable from the plunger rod.

Optionally, when the plunger rod is located along the first range of motion relative to the syringe, the blocking element is secured to the plunger rod, such that it cannot be removed from the plunger rod.

Optionally, when the plunger rod is located along the subsequent range of motion relative to the syringe, the blocking element is not secured to the plunger rod, such that it can be removed from the plunger rod.

The blocking element may only be removable from the plunger rod at the second region. The plunger rod may be slidable relative to the blocking element. The plunger rod may be slidable though an aperture in the blocking element.

The blocking element may be secured to the plunger rod when the plunger rod is in a first position relative to the syringe and the blocking element may be removable from the plunger rod when the plunger rod is in a second position relative to the syringe.

The first position may be an initial position of the plunger rod and the second position of the plunger rod may be after the plunger rod has moved over the first range of motion

The blocking element may be secured to the syringe whilst the plunger rod is located along the first range of motion and may be removable from the syringe when the plunger rod has completed the first range of motion. This may help prevent the blocking element being removed from the device until a first dose of medicament has been expelled and thereby help prevent incorrect use of the device or incorrect dosage delivery.

The blocking element may comprise a dose clip. The dose clip/blocking element may comprise a rigid component. That is, the dose clip may comprise a substantially rigid body. The dose clip may comprise a unitary body. The dose clip/blocking element may be removable from the plunger rod without deflection of a part of the blocking element/dose clip.

The blocking element may comprise a locking formation configured to fix the blocking element relative to the syringe whilst the plunger rod is moveable over the first range of motion. This may further help avoid incorrect use of the device by the blocking element being moved along the plunger rod before the first dose of medicament has been expelled.

The locking formation may comprise one of a rib or groove, arranged to be in sliding engagement with the other of a groove or rib formed on the syringe.

The syringe may comprise a component at, attached to, or configured to attach to, a proximal end of the syringe. The component of the syringe may be a separate component, or may be a part integrally formed with the syringe. The syringe may comprise a lip at a proximal end thereof, and the component may be configured to attach to the lip of the syringe. The component provided at or attached to the proximal end of the syringe may comprise a finger flange. The finger flange may be releaseably or detachably attached to the syringe. This may enable the blocking element and/or finger flange to be fitted to known configurations of syringe, and/or retro-fitted to such syringes.

The other of the groove and rib may be formed on a finger flange secured to the syringe.

The locking formation may permit the blocking element to be slid off the syringe in a direction perpendicular to the direction of movement of the plunger rod. The blocking element may be removable from the syringe in a direction substantially perpendicular to an axis of the elongate plunger rod. This may facilitate ease and intuitive operation by a user of the device.

The syringe may comprise a barrel and an outlet at a distal end of the barrel.

The syringe may comprise a piston in sealing contact with an inside wall of the chamber and slidable within the chamber by movement of the plunger rod to expel medicament from the outlet.

The blocking element may include an aperture and an opening on a side of the blocking element in communication with the aperture, and the plunger rod may be slidable through the aperture in the blocking element.

The blocking element may be removable from the plunger rod in the second region may not be removable from the plunger rod in the first region.

The first region may not be able to pass through the opening in blocking element, preventing removal of the blocking element from plunger rod, and wherein the second region may be able to pass through the opening permitting removal of blocking element from plunger rod. This may help avoid removal of the blocking element until the first dose has been expelled, helping prevent incorrect use of the device.

The second region may be able to pass through the opening in a direction substantially perpendicular to an axis of the aperture in the blocking element.

The first region may be a region of a first outer dimension, and the second region may be a region of a second outer dimension that is smaller than the first outer dimension.

The plunger rod may be an elongate element that is substantially circular, square, hexagonal, other geometric shape in cross-section.

The blocking element may include a grip region to facilitate removal of the blocking element from the plunger rod and/or the syringe.

The grip region may comprise a projecting tab or a region of increased surface roughness or enhanced friction surface.

The plunger rod and blocking element may be configured such that substantially half of the medicament in the chamber is expelled as the plunger rod moves over the first range of motion, and substantially half of the medicament in the chamber is expelled as the plunger rod moves over a second range of motion.

The medicament delivery system may further comprise an atomiser at the outlet of the syringe. Such atomiser may facilitate insertion of the atomiser and outlet into an orifice of a patient into which the medicament is to be delivered. The atomiser and/or outlet 16 may comprise a luer lock to sealingly connect the atomiser to the outlet. The medicament delivery system may comprise atomiser, plug, adaptor or other component at the outlet of the syringe. Such component at the outlet may comprise an atomiser or may comprise a needle. A needle may be provided if medicament is intended to be delivered in doses into a patient in a particular manner. Such delivery may include intravenous delivery, subcutaneous delivery, and/or intramuscular delivery. The needle diameter may vary within the scope of the present disclosure, and may be selected according to the desired form of medicament delivery.

An atomiser, plug, adaptor or other component may be configured to atomise a liquid medicament as the medicament is expelled through the outlet and through a medicament passage in the atomiser, plug, adaptor or other component.

The system may be configured to deliver more than two doses of medicament. The system may be configured to divide the medicament into more than two doses, and may be configured to divide the medicament delivery into three, four, five, six or more doses. The system may comprise a plurality of blocking elements, and may comprise two, three, four, five, six or more blocking elements. Each blocking element may be provided around the plunger rod. The blocking elements may be initially disposed adjacent each other in a longitudinal direction of the plunger rod.

At least one blocking element may comprise a locking formation configured to fix the at least one blocking element to an adjacent blocking element whilst the plunger rod is moveable over a second or subsequent range of motion. The locking formation may be configured to fix the at least one blocking element to an adjacent blocking element in an axial direction of the plunger rod. This may further help avoid incorrect use of the device by a blocking element being moved along the plunger rod and/or removed before the second or subsequent dose of medicament has been expelled. Two or more, or all blocking elements may comprise such engaging or engageable locking formations.

The locking formation may comprise one of a rib or groove, arranged to be in sliding engagement with the other of a groove or rib formed on an adjacent blocking element.

Medicament may be received within the chamber of the syringe.

The medicament may any medicament described herein. The medicament may be a vaccine adapted for nasal administration. The vaccine may be an RSV vaccine.

The system may comprise an intranasal atomization delivery device.

The system may be configured to deliver about ½ of a dose to each nostril.

The system may be configured to deliver about 0.2 mL, wherein about 0.1 mL is delivered to each nostril.

The intranasal atomization delivery system may deliver an average droplet size Dᵥ₅₀ of about 10-120 μm.

The intranasal atomization delivery system may deliver an average droplet size Dᵥ₅₀ delivered to each nostril may be about 10-120 μm, about 30-110 μm, about 50-110 μm, about 70-110 μm, or about 80-110 μm.

The intranasal atomization delivery system may be configured so that an average shot volume to each nostril is between about 85 μL to about 120 μL, about 90 μL to about 115 μL, or about 95 μL to about 115 μL.

Also provided is an apparatus comprising a finger flange, a blocking element and a plunger rod for use with a syringe, the plunger rod receivable within an aperture in the blocking element, wherein the plunger rod comprises a first region of a first outer configuration and a second region of a second outer configuration that is different to the first outer configuration, the apparatus being attachable to the syringe such that the plunger rod is slidable within and relative to the syringe, and such that the blocking element permits the plunger rod to move over a first range of motion relative to the syringe to expel a first dose of medicament, and blocks the plunger rod from moving over a subsequent range of motion relative to the syringe to expel a further dose of medicament.

The blocking element may be removable from the finger flange and the plunger rod to permit the plunger rod to be free to be moved over a subsequent range of motion relative to the syringe to expel further medicament.

Optionally, when the plunger rod is located along the first range of motion relative to the syringe, the blocking element permits the plunger rod to move over the first range of motion, and the blocking element becomes removable from the plunger rod once the plunger rod has completed the first range of motion.

Optionally, when the plunger rod is located along the first range of motion relative to the syringe, the blocking element permits the plunger rod to move over the first range of motion and the blocking element is not removable from the plunger rod; and the blocking element is removable from the plunger rod once the plunger rod has completed the first range of motion.

Optionally, when the plunger rod is located along the first range of motion relative to the syringe, the blocking element is not removable from the plunger rod; and when the plunger rod is located along the subsequent range of motion relative to the syringe, the blocking element is removable from the plunger rod.

Optionally, when the plunger rod is located along the first range of motion relative to the syringe, the blocking element is not removable from the plunger rod.

Optionally, when the plunger rod is located along the subsequent range of motion relative to the syringe, the blocking element is not removable from the plunger rod.

Optionally, when the plunger rod is located along the first range of motion relative to the syringe, the blocking element is secured to the plunger rod, such that it cannot be removed from the plunger rod.

Optionally, when the plunger rod is located along the subsequent range of motion relative to the syringe, the blocking element is not secured to the plunger rod, such that it can be removed from the plunger rod.

Also provided is a method of operating a medicament delivery system comprising a syringe comprising a chamber for containing medicament and an outlet at a distal end of the syringe, a plunger rod mounted within the syringe and slidable relative to the syringe to cause medicament to be expelled from the chamber when medicament is within the chamber, wherein the plunger rod comprises a first region of a first outer configuration and a second region of a second outer configuration that is different to the first outer configuration, and a blocking element configured to be arranged at a proximal end of the syringe and disposed around the plunger rod, the method comprising, moving the plunger rod over a first range of motion relative to the syringe to expel a first dose of medicament, the blocking element blocking the plunger rod from moving over a subsequent range of motion relative to the syringe, removing the blocking element from plunger rod, and moving the plunger rod over the subsequent range of motion relative to the syringe to expel further dose of medicament.

Optionally, when the plunger rod is located along the first range of motion relative to the syringe, the blocking element permits the plunger rod to move over the first range of motion, and the blocking element becomes removable from the plunger rod once the plunger rod has completed the first range of motion.

Optionally, when the plunger rod is located along the first range of motion relative to the syringe, the blocking element permits the plunger rod to move over the first range of motion and the blocking element is not removable from the plunger rod; and the blocking element is removable from the plunger rod once the plunger rod has completed the first range of motion.

Optionally, when the plunger rod is located along the first range of motion relative to the syringe, the blocking element is not removable from the plunger rod; and when the plunger rod is located along the subsequent range of motion relative to the syringe, the blocking element is removable from the plunger rod.

Optionally, when the plunger rod is located along the first range of motion relative to the syringe, the blocking element is not removable from the plunger rod.

Optionally, when the plunger rod is located along the subsequent range of motion relative to the syringe, the blocking element is not removable from the plunger rod.

Optionally, when the plunger rod is located along the first range of motion relative to the syringe, the blocking element is secured to the plunger rod, such that it cannot be removed from the plunger rod.

Optionally, when the plunger rod is located along the subsequent range of motion relative to the syringe, the blocking element is not secured to the plunger rod, such that it can be removed from the plunger rod.

The method may comprise expelling/administering an RSV vaccine.

The delivery system may comprise an intranasal atomisation delivery device.

The RSV vaccine may be expelled/delivered intranasally with about ½ dose delivered to each nostril.

The intranasal dose of the RSV vaccine may be delivered in about 0.2 mL, wherein about 0.1 mL is delivered to each nostril.

The method may comprise intranasal atomization delivery of an average droplet size Dᵥ₅₀ of about 10-120 μm.

The method may comprise intranasal atomization delivery of an average droplet size Dᵥ₅₀ delivered to each nostril may be about 10-120 μm, about 30-110 μm, about 50-110 μm, about 70-110 μm, or about 80-110 μm.

The method may comprise intranasal atomization delivery so that an average shot volume to each nostril is between about 85 μL to about 120 μL, about 90 μL to about 115 μL, or about 95 μL to about 115 μL.

The method may initially comprise inserting the outlet into a first nostril of a patient. The method may comprise inserting the outlet into a second nostril of a patient after removing the blocking element from the device and before moving the plunger rod over the subsequent range of motion.

The method may comprise expelling/delivering more than two doses of medicament. The method may comprise dividing the medicament into more than two doses, and may comprise dividing the medicament into three, four, five, six or more doses. The method may comprise providing and sequential removal from the plunger rod, of a plurality of blocking elements, and may comprise two, three, four, five, six or more blocking elements.

The plunger rod may define a longitudinal axis. The plunger rod axis may be aligned with a longitudinal axis of the syringe. The method may comprise the plunger rod moving in an axial direction relative to the or each blocking element. The plunger rod may be moveable in an axial direction until an end surface of the plunger rod abuts an adjacent blocking element. The blocking element may be disposed between a surface of the plunger rod and a proximal end of the syringe after delivery/expulsion of each dose of medicament.

According to the present disclosure, there is provided a medicament delivery system comprising a syringe comprising a chamber for containing medicament and an outlet at a distal end of the syringe through which medicament can be expelled from the chamber, a plunger rod mounted within the syringe and slidable relative to the syringe to cause medicament to be expelled from the chamber when medicament is within the chamber, wherein the plunger rod comprises a first region of a first outer configuration and a second region of a second outer configuration that is different to the first outer configuration, and a blocking element at a proximal end of the syringe and disposed around the plunger rod, and which permits the plunger rod to move over a first range of motion relative to the container to expel a first dose of medicament, and blocks the plunger rod from moving over a subsequent range of motion relative to the container to expel further medicament, wherein the blocking element is removable from the plunger rod, and wherein the plunger rod is free to be moved over the subsequent range of motion relative to the container to expel further medicament once the blocking element is removed from the device. These and other aspects of the invention will be apparent from and elucidated with reference to the embodiments described hereinafter.

### BRIEF DESCRIPTION OF THE FIGURES

Embodiments will now be described, by way of example only, with reference to the accompanying drawings, in which:

FIG. 1 is a perspective view of a medicament delivery device of a first embodiment, shown in an initial position;

FIG. 2 is a side view of the device of FIG. 1;

FIG. 3 is a side view of the device of FIGS. 1 and 2 after the plunger rod has been moved over a first range of motion;

FIG. 4 is a side view of the device of FIG. 3 and a user removing a dose divider from the device;

FIG. 5 is a side view of the device of FIG. 3 once the dose divider has been removed;

FIG. 6 is a side view of the device of FIG. 5 after the plunger rod has been moved over a second range of motion;

FIG. 7 is a perspective view of the dose divider of the device of FIGS. 1 to 6;

FIG. 8 is an enlarged partial side view of a portion of the device of FIGS. 1 to 6 showing the medicament container and dose divider, and the locking formations enabling attachment of the dose divider to the medicament container;

FIG. 9 is a perspective view of the plunger rod of the device of FIGS. 1 to 6, with optional piston attached;

FIG. 10 is a cross-sectional view of the device of FIGS. 1 to 6 along the line A-A of FIG. 2 showing dimensions of first and second regions of the plunger rod;

FIG. 11 is a perspective view of an alternative configuration of plunger rod and dose divider;

FIG. 12 is a perspective view of yet another alternative configuration of plunger rod and dose divider;

FIG. 13 is an exploded perspective view of an apparatus comprising a blocking element, plunger rod and finger flange attachment of an embodiment, together with a portion of a syringe to which they connect;

FIG. 14 is a cross-sectional view of the apparatus of FIG. 13;

FIG. 15 is a side view of a medicament delivery device of another embodiment, shown in an initial position;

FIG. 16. Is a side view of the device of FIG. 15 after the plunger rod has been moved over a first range of motion;

FIG. 17 is a side view of the device of FIG. 16 once a first dose divider has been removed;

FIG. 18 is a side view of the device of FIG. 17 after the plunger rod has been moved over a second range of motion;

FIG. 19. Is a side view of the device of FIG. 18 after a second dose divider has been removed;

FIG. 20. Is a side view of the device of FIG. 19 after the plunger rod has been moved over a third range of motion.

### DETAILED DESCRIPTION

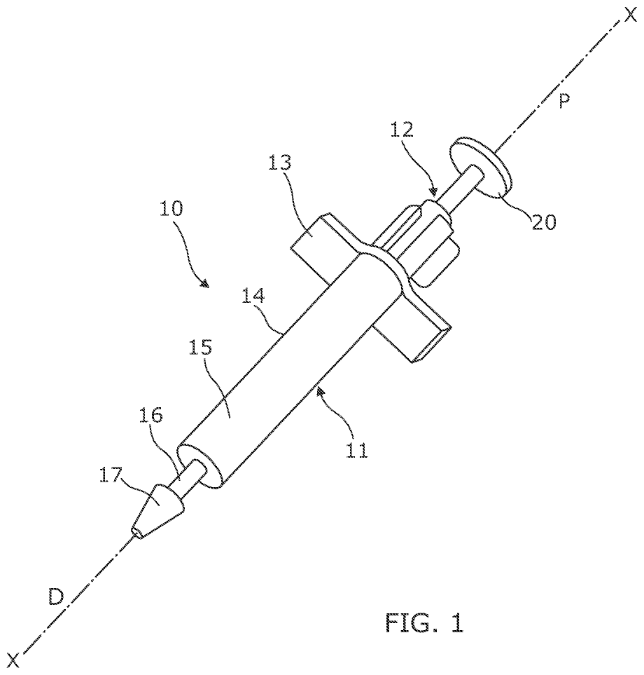

FIGS. 1 to 6 show a medicament delivery system or device 10 (hereafter “device 10”) according to a first embodiment. The device 10 has a proximal end P and a distal end D, and is generally elongate with a central axis X-X. The device 10 comprises a medicament container 11 in the form of a syringe (hereafter “container 11”), an actuator 12 in the form of a plunger rod or piston rod 12, and finger flange 13 secured to a proximal end of the container 11. The container 11 is elongate and comprises an outer side wall 14 defining a medicament chamber 15. The container 11 is open at a proximal end P and includes an outlet 16 at the distal end D opposite to the proximal end of the container 11. The term “distal” refers to a location that is relatively closer to the outlet 16 and from which medicament is delivered in use, and the term “proximal” refers to a location that is relatively further away from the outlet 16. A distal region of the plunger rod 12, received within the container 11, is in sealing contact with the inside wall of the container 11. The proximal end of the plunger rod 12 includes an end surface 20 which, in use, a user would press a thumb against to dispense medicament (described in more detail below).

The outlet 16 comprises a narrowing of the container into a nozzle. An atomiser 17 is provided on the outlet 16. The atomiser 17 and/or outlet 16 may comprise a luer lock to sealingly connect the atomiser 17 to the outlet 16. In some embodiments, the atomiser 17 may be formed to facilitate insertion of the outlet into an orifice into which medicament is to be delivered. Optionally, the atomiser 17 may be configured with a medicament passage configured to atomise a liquid medicament expelled through the outlet 16 and through the atomiser 17.

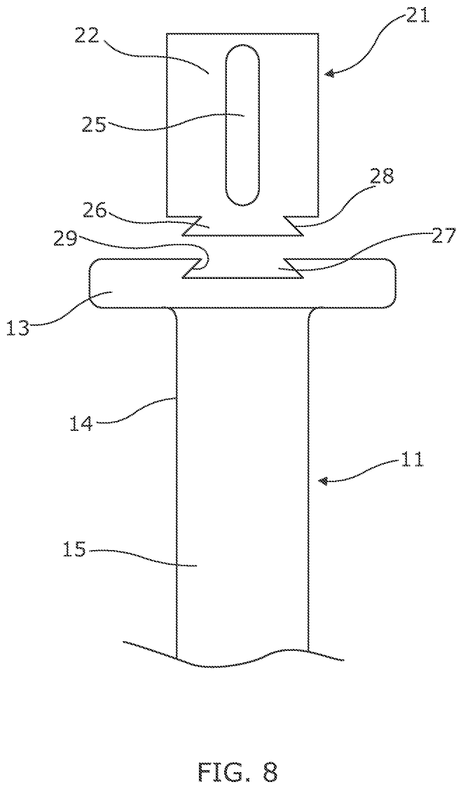

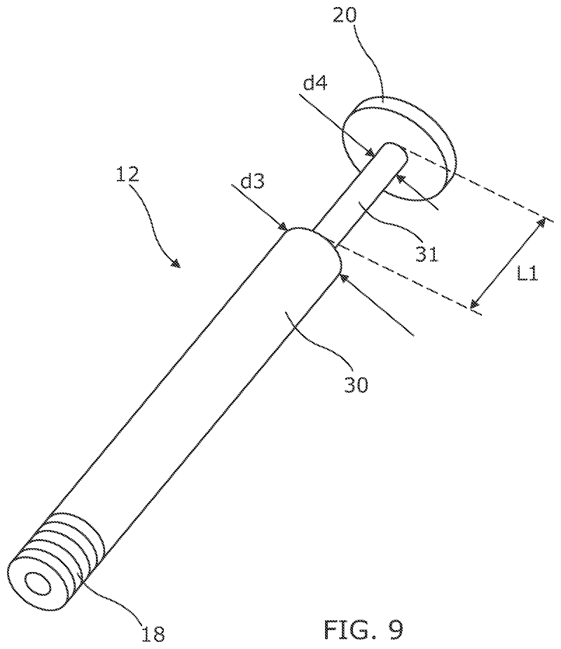

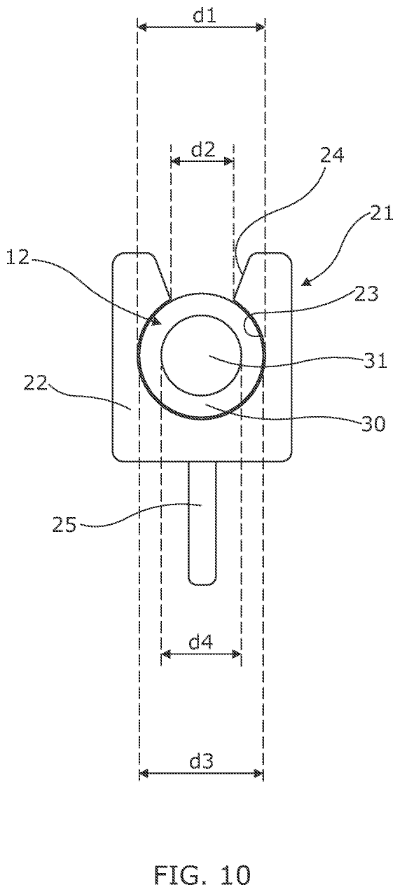

The container described herein may be any suitable shape or configuration, such as a hollow tube or other body. In the exemplary embodiment shown in FIGS. 1 to 6, the device 10 is a syringe comprising a syringe barrel and the plunger rod 12 is received within the syringe barrel. In some embodiments, a piston 18 may be provided within the syringe chamber 15 in sealing contact with the inside wall of the chamber 15. In such an arrangement, the piston 18 may be attached to the distal end of the plunger rod 12 (see FIG. 9), and the plunger rod 12 may therefore not need to make sealing contact with the inside wall of the container 11.

In use, medicament 19 would be provided within the medicament chamber 15, and would remain in the chamber 15 due to the seal of the plunger rod 12 and/or piston 18 at the proximal end of the chamber 15, and the narrow outlet 16 at the distal end of the chamber 15, which may be provided with a cap, valve, temporary rupturable seal, or other closure. Alternatively, the narrow outlet 16 alone may prevent medicament 19 escaping from the distal end of the chamber 15. The medicament 19 may be expelled in one or more doses from the chamber 15 by linearly moving the plunger rod 12 in a distal direction into the chamber 15 to reduce the volume of the chamber and force the medicament 19 out of the outlet 16.

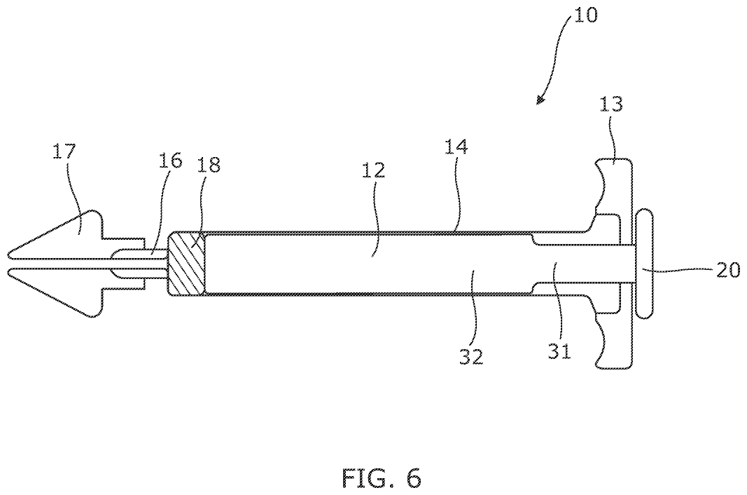

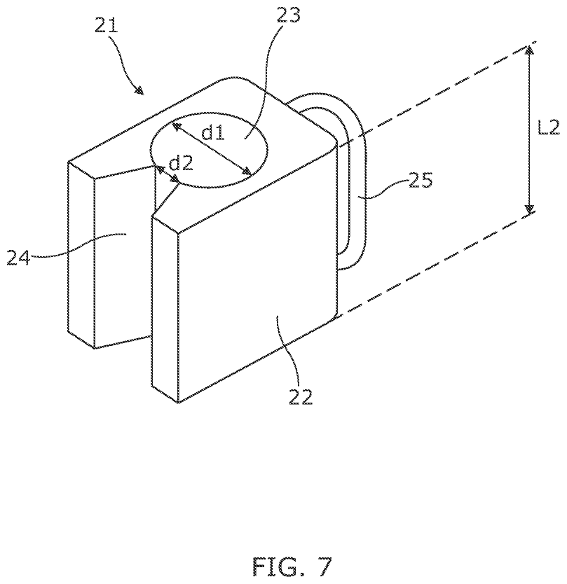

The device 10 includes a blocking element 21 which functions as a “dose divider” in use of the device. The blocking element 21 may be referred to as a “dose clip”. The blocking element, dose divider, or dose clip 21 (“blocking element” hereafter) is shown in more detail in FIG. 7 and comprises a body 22 with an aperture 23 extending through the body 22. An opening 24 is formed in one side of the body 22 and extends to the aperture across the length of the aperture 23 from one side of the body 22 to the opposite side so that the aperture is open along its length via the opening 24. The opening 24 is narrower than the diameter of the aperture 23 over at least part of the opening, as shown in FIG. 7 by dimension d2 being less than dimension d1. In the embodiment shown in FIG. 7, the opening 24 comprises a slot which tapers outwardly from the aperture 23 towards the outer side of the body 22. However, in alternative embodiments, the opening 24 may be of a substantially constant width between the aperture 23 and the outer side of the body 22. The blocking element 21 may optionally include a grip region 25. In the embodiment shown, the grip region 25 comprises a projecting tab. However, in alternative embodiments, the grip region 25 may comprise a material, coating, or surface formation to enhance grip or friction, such as a roughened or textured surface around the body 22.

The blocking element 21 is provided with a locking formation 26 formed on a distal-facing surface of the body 22. These can be seen in FIG. 8. The finger flange 13 is provided with a cooperating locking formation 27 which is configured to engage and lock with the locking formation on the blocking element 21. In the exemplary embodiment shown, these locking formations comprise a projecting rib with outwardly-tapering edges 28 which is formed on the blocking element 21, and a corresponding-shaped groove with outwardly tapering slot edges 29 formed in the finger flange 13. As will be appreciated from FIG. 8, the rib 26 on the blocking element 21 can be slid into the groove 27 on the finger flange 13 to fixedly secure the blocking element 21 to the finger flange 13. In the exemplary embodiment shown, the blocking element 21 is slid onto the finger flange 13 in a direction substantially perpendicular to the axial direction of the device 10. The blocking element 21 is thereby secured to the finger flange 13 to prevent relative movement in the axial direction of the device 10-that is, to prevent the blocking element 21 from being moveable in a proximal direction away from the finger flange 13.

Although the exemplary embodiment shown in FIG. 8 comprises the locking formations 26, 27 as a cooperating tapered rib and groove, alternative embodiments may comprise different configurations of locking formations arranged to axially secure the blocking element 21 to the finger flange 13, but to permit the blocking element 21 to be slid off the finger flange 13 in a direction substantially perpendicular to the axial direction of the device 10, such as “L” or “T”-shaped projections, or any other non-axially linear projection, and correspondingly-shaped grooves. Also, a projecting element may be formed on either of the blocking element 21 and finger flange 13, and a corresponding groove, slot or recess formed in the other of the finger flange 13 and blocking element 21. Furthermore, in other embodiments, the finger flange 13 may be omitted and the locking formation 27 may be formed in the proximal region of the container 11. In yet further alternative embodiments, the device 10 may omit the locking formations altogether.

The plunger rod 12 is shown in more detail in FIG. 9 and comprises an elongate rod, and in FIG. 9 is shown with an optional piston 18, so the plunger rod 12 serves as a piston rod or plunger rod for moving the piston 18 within the container 11. In various embodiments, the piston 18 may be formed integrally with the plunger rod 12, or may be mechanically or otherwise (such as by adhesive bonding) attached to the plunger rod 12, or the plunger rod 12 may abut against the piston 18. The plunger rod 12 comprises a first region 30 of a first outer configuration or dimension, and a second region 31 of a second configuration or outer dimension. The second region 31 is disposed in an axial direction between the first region 30 and the end surface 20. FIG. 9 shows the first region having an outer dimension d3, and the second region having an outer dimension d4, where d3 is greater than d4.

In the exemplary embodiment shown in FIG. 9, the plunger rod 12 is substantially circular in cross-section so the dimensions d3 and d4 correspond to the diameter of the plunger rod 12 at those regions. However, it is intended that other configurations of plunger rod may be provided and which may have different cross-sectional configurations, such as oval, square, triangular, hexagonal, or other polygonal shapes.

The plunger rod 12 is arranged to slide through the aperture 23 in the blocking element 21 once the blocking element 21 is secured to the finger flange 13 by way of the locking formations 26, 27 as described above. When the blocking element 21 is secured to the finger flange 13, the aperture 23 is aligned with the axis of the bore of the medicament chamber 15, so the plunger rod 12 extends through the aperture 23 and into the container 11. The aperture 23 is configured such that d1 is slightly larger than d3 to allow the plunger rod 12 to freely slide through the aperture 23. Furthermore, the dimension d3 of the first region 30 of the plunger rod 12 is greater than the dimension d2 such that the first region 30 cannot pass through the opening 24. However, the second region 31 of the plunger rod 12 is configured such that the second region 31 is able to pass through the opening 24. This may be by dimension d4 of the second region 31 being smaller than dimension d2 of the opening 24. Alternatively, the dimension d4 of the second region may be substantially the same as dimension d2 of the opening 24, or slightly greater than dimension d2, and second region 31 may be able to pass through the opening 24 by a friction fit and a predetermined force being exerted on the plunger rod 12/blocking element 21, and/or by slight deflection of the body 22 to allow the opening 24 to widen slightly to permit the second region 31 to pass through the opening 24.

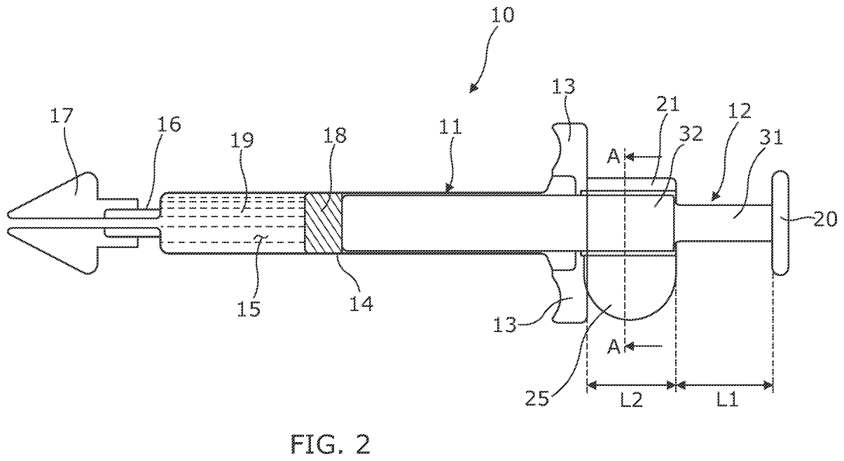

Operation of the device 10 of the above-described embodiment will now be described. The operation of the device 10 will be described in the context of use as an intranasal medicament delivery device 10, that is, a device for delivery of medicament into the nostril(s) of a patient. However, the concept is not intended to be limited to intranasal devices and may equally be applicable to various other forms of medicament delivery devices in which the medicament is intended to be delivered in two or more separate doses. In the context of an intranasal device, the volume of medicament in the device 10, for example a vaccine, is often required to be delivered into each nostril, often with the volume of medicament divided equally into a dose for each nostril. In use of such a device, the device 10 starts in the initial position shown in FIG. 2. Here, the plunger rod 12 is in its start position and none of the medicament 19 has been expelled from within the chamber 15. Also in this initial position, the blocking element 21 is secured to the finger flange 13 by the locking formations 26, 27, and is unable to be slid off the finger flange 13 since the first region 30 of the plunger rod 12 is located in the aperture 23 and so cannot pass through the opening 24.

The user (which may be a patient them self if self-administered, or a healthcare professional or other user if the medicament is being administered to the patient by a third party) inserts the atomiser 17 into the first nostril. The user then presses the end surface 20 with their thumb, with their index and middle fingers on the finger flange 13, to move the plunger rod 12 axially in a distal direction D. This pushes the piston 18 distally within the chamber 15 and expels the medicament 19 out of the outlet 16 and through the medicament passage of the atomiser 17 to atomise the medicament for inhalation by the patient. The user continues to move the plunger rod 12 distally over this first range of motion until the distal face of the end surface 20 abuts the proximal face of the blocking element 21 and the plunger rod 12 therefore cannot move any further in the distal direction. At this point, the plunger rod 12 is in an intermediate position and a first dose of the medicament 19 has been delivered to the patient. Also at this point, first region 30 of the plunger rod 12 has moved into the container 11 and now the second region 31 of the plunger rod 12 is positioned within the aperture 23 of the blocking element 21. Since the dimension d4 of the second region 31 of the plunger rod 12 and the dimension d2 of the opening 24 in the blocking element 21 are configured as described above, namely that the second region 31 of the plunger rod 12 can pass through the opening 24, the blocking element 21 is no longer locked to the finger flange 13/device 10.

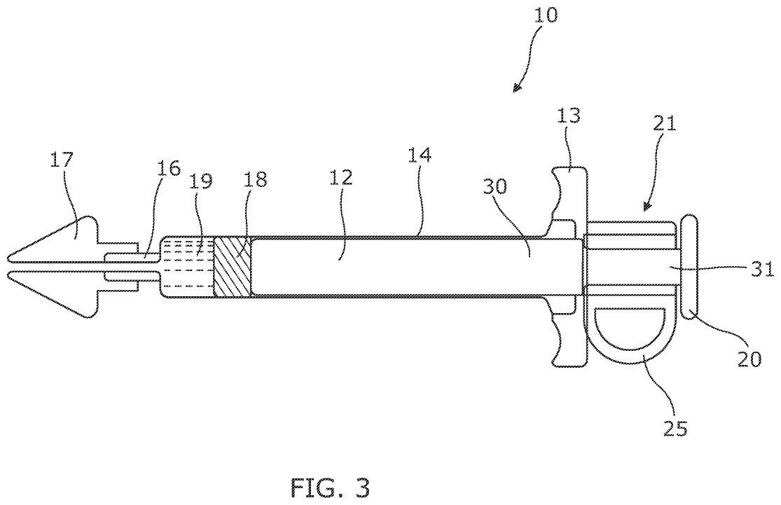

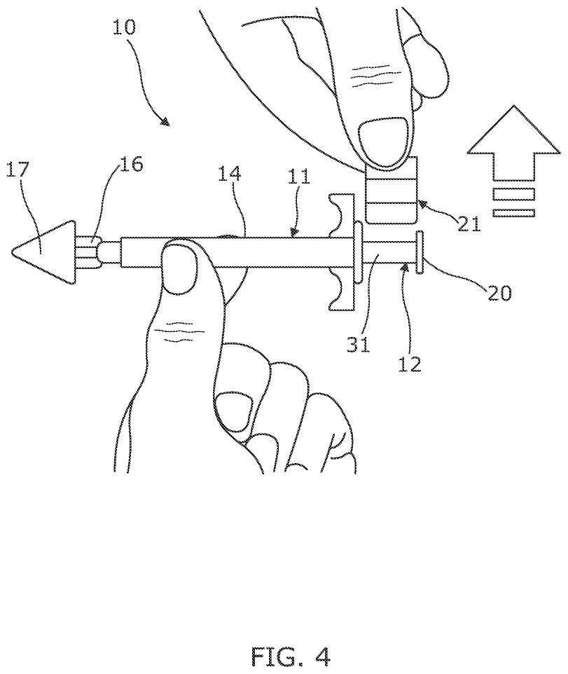

The user then removes the device 10 from the first nostril and slides the blocking element 21 off the device 10 as shown in FIG. 4, helped by gripping the grip region 25, so that the device 10 is in the configuration as shown in FIG. 3. That is, the plunger rod 12 is in an intermediate position having been moved over a first range of motion but there remains some medicament 19 within the chamber 15 for subsequent dose(s) to be delivered. The user then inserts the atomiser 17 into the other nostril. Since the blocking element 21 has been removed, the plunger rod 12 is no longer prevented from moving further axially in the distal direction. The user then presses the end surface 20 again to move the plunger rod 12 axially in a distal direction D which pushes the piston 18 distally within the chamber 15 and expels the remaining medicament 19 out of the outlet 16 and through the medicament passage of the atomiser 17 to atomise the medicament for inhalation by the patient. The user continues to move the plunger rod 12 distally over this second range of motion until either the distal face of the end surface 20 abuts the finger flange 13, or the piston 18 abuts the distal end of the chamber 15 and therefore cannot move any further in the distal direction. At this point, the plunger rod 12 is in an end position and the remaining medicament 19 has been expelled as a second and final dose delivered to the patient.

The device 10 described above provides a simple and effective way to ensure a user is able to divide the medicament delivery process into two separate doses, and the blocking element 21 provides an effective way to ensure the medicament 19 is divided as intended and the user cannot accidentally deliver too much/all of the medicament in the first dose. Furthermore, since the blocking element 21 is not removable from the finger flange 13 until the plunger rod 12 has moved over the first range of motion, the blocking element 21 cannot be tampered with and removed before the first dose of medicament 19 is delivered. This further helps towards ensuring the medicament is delivered in the intended dose volumes in each dose.

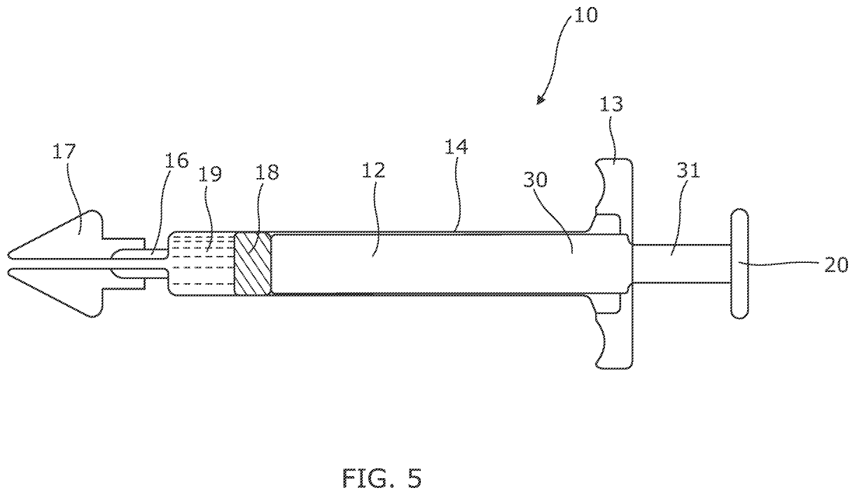

As can be seen from FIGS. 2, 7 and 9, the distance between the proximal end of the blocking element 21 and the distal face of the end surface 20 has a length dimension in the axial direction of L1. In the exemplary embodiment shown, this length L1 also corresponds to the length in the axial direction of the second region 31 of the plunger rod 12. Also, the blocking element 21 has a length dimension in the axial direction of L2. It will be appreciated that the volume of medicament 19 delivered in the first dose is determined by the distance the plunger rod 12 is able to move from the initial position (shown in FIG. 2) to the intermediate position (shown in FIGS. 3 and 4), which is limited by when the end surface 20 abuts the blocking element 21. That is, this volume of medicament of the first dose is determined by the distance L1 over which the plunger rod 12 moves in the first range of motion. It will also be appreciated that the volume of medicament 19 delivered in the second dose is determined by the remaining distance the plunger rod 12 is able to move from the intermediate position (shown in FIGS. 3 and 4) to the end position (shown in FIG. 6), which is limited by when the end surface 20 abuts the finger flange 13, which may also coincide with when the piston 18 abuts the distal end of the chamber 15. That is, this volume of medicament of the second dose is determined by the distance L2 over which the plunger rod 12 moves in the second range of motion.

In view of the above, the ratio of volumes of first and second doses of medicament to be delivered can be controlled by controlling the first and second ranges of motion of the plunger rod 12, which may be controlled by selecting dimensions L1 and L2 of the device 10 as necessary. In embodiments where the first and second doses are to be equal, it will be appreciated that dimensions L1 and L2 can be made the same.

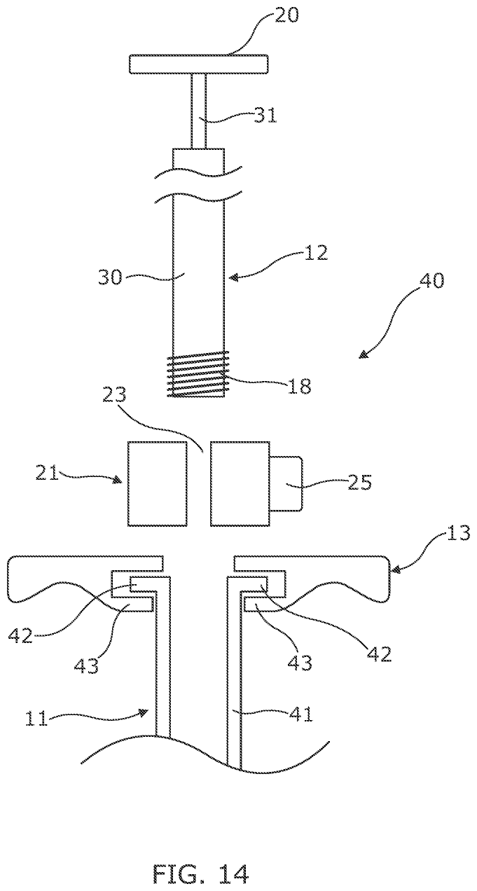

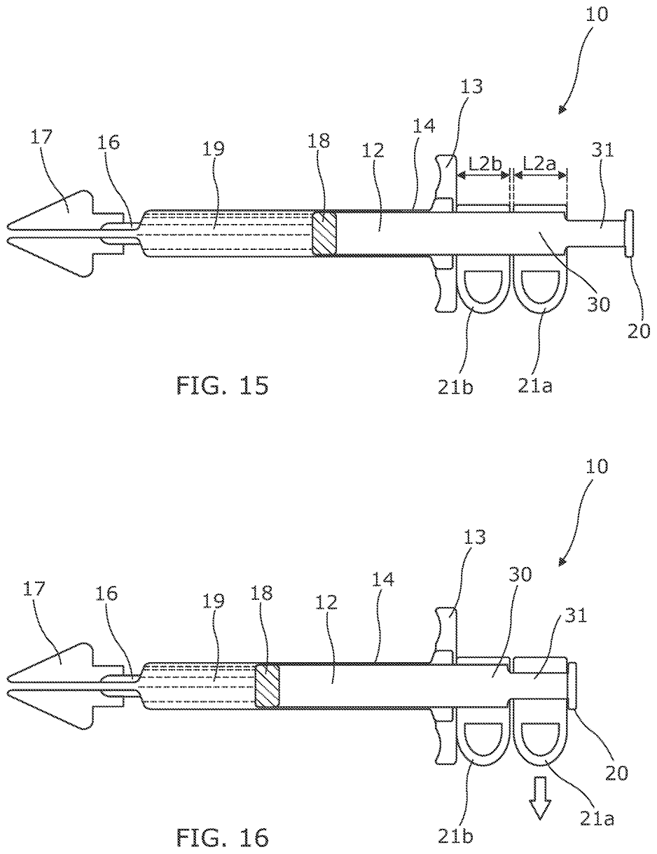

It shall be appreciated that the present disclosure may be configured to deliver more than two doses of medicament. For example, the plunger rod 12 shown in FIG. 9 could be adapted to allow for the controlled delivery of three or more doses by the inclusion of additional regions of reduced diameter between the first region 30 and the second region 31. Each additional region could comprise a reduced diameter to the adjacent region in the distal direction. A plurality of blocking elements 21 could be provided with appropriately sized apertures 23 corresponding to each additional region of plunger rod diameter, such that each blocking element 21 is reached and abutted per dose delivery, and requires removal before the next dose can be delivered. Such an alternative embodiment is shown and described in more detail below with reference to FIGS. 15 to 20.

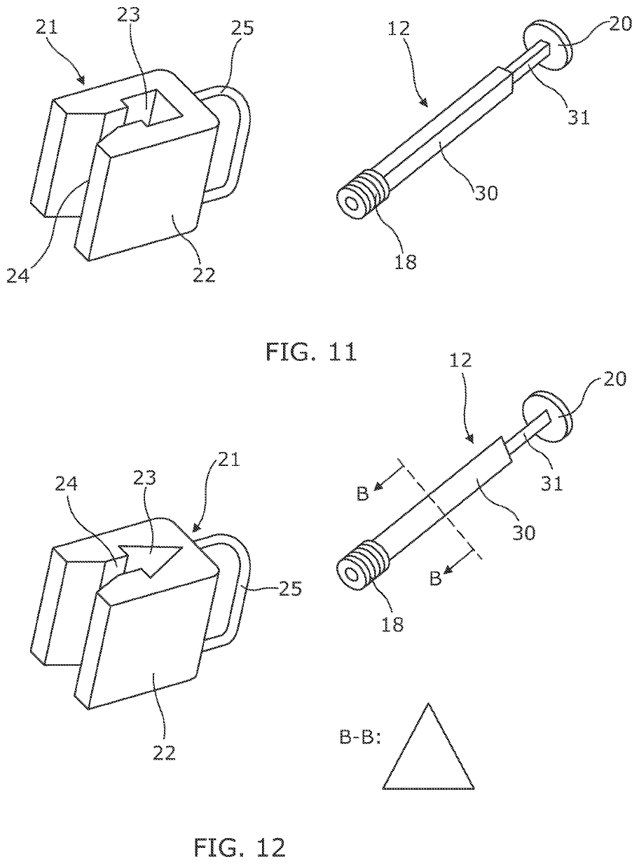

FIGS. 11 and 12 show an apparatus 40 of an embodiment, configured for attachment to a medicament container 11 such as a syringe. Like features from the previously-described embodiments retain the same reference numerals. It is intended that the apparatus 40 can be attached to a standard syringe having a barrel 41 and a lip 42 projecting outwardly around the edge of the barrel 41 at the proximal end thereof. Thereby, the apparatus 40 can be supplied as a separate kit to be fitted to conventional syringes, or retro-fitted to such syringes, before or after assembly and/or filling with a medicament.

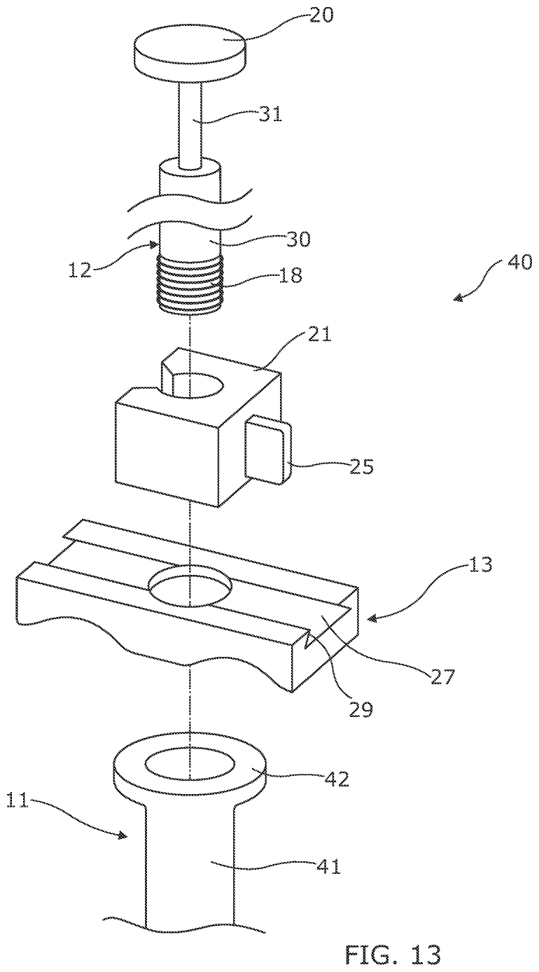

The apparatus 40 comprises a finger flange 13, a blocking element 21 and a plunger rod 12, all as previously described. The finger flange 13 includes projections 43 (see FIG. 14) which can locate distally of the lip 42 to secure the finger flange 13 to the medicament container/syringe 11. Thereafter, once secured to a medicament container/syringe 11, operation of the medicament delivery device 10 is as previously described, so will not be repeated.

Although the above-described embodiments comprise a plunger rod 12 which is circular in cross-section, with a corresponding circular aperture 23 in the blocking element 21, other configurations of plunger rod 12 and aperture 23 are intended, such, for example, square or triangular, as shown in FIGS. 14 and 15 (in which like features of plunger rod 12 and blocking element 21 to those described previously retain the same reference numerals).

FIGS. 15 to 20 show a further embodiment which is configured to allow for the controlled delivery of more than two doses of medicament. In the exemplary embodiment shown in FIGS. 15-20, the device 10 is configured to provide for the controlled delivery of three doses of medicament. However, in other embodiments envisaged within the scope of the present disclosure, devices configured to allow for the delivery of more than three doses of medicament are intended. In the further embodiment shown in FIGS. 15-20, like features in common with the previously-described embodiments retain the same reference numerals and a detailed description thereof will not be repeated.

FIG. 15 shows a side view of the device 10 of the further embodiment in an initial position. It can be seen that the device 10 comprises a medicament container 11 in the form of a syringe (hereafter “container 11”), an plunger rod 12 having an end surface 20, a finger flange 13, an outlet 16 provided with an atomiser 17, as described previously.

In use, medicament 19 would be provided within the chamber 15, and would remain in the chamber 15 due to the seal of the plunger rod 12 and/or piston 18 at the proximal end of the chamber 15, and the narrow outlet 16 at the distal end of the chamber 15, which may be provided with a cap, valve, temporary rupturable seal, or other closure.

A difference with the embodiment of FIGS. 15-20 is that the device 10 includes two blocking elements 21a, 21b, a first blocking element 21a and a second blocking element 21b. Each blocking element 21a, 21b functions as a “dose divider” in use of the device. As mentioned previously, the blocking elements 21a, 21b may be referred to as a “dose divider” or “dose clip” herein. The blocking elements 21a, 21b are the same are described previously with reference to FIG. 7 and so detailed description thereof will not be repeated.

Operation of the device 10 of the embodiment of FIGS. 15-20 will now be described. The operation of the device 10 will be described in the context of use as an intranasal medicament delivery device 10, that is, a device for delivery of medicament into the nostril(s) of a patient. However, the concept is not intended to be limited to intranasal devices and may equally be applicable to various other forms of medicament delivery devices in which the medicament is intended to be delivered in two or more separate doses.

In the context of an intranasal device, the volume of medicament in the device 10, for example a vaccine, is often required to be delivered into each nostril, often with the volume of medicament divided equally into a dose for each nostril. However, the device 10 may be intended to be used for multiple patients, and so the volume of medicament may be intended to be delivered into more than two doses. With reference to FIGS. 15-20, the device will be described as dividing the volume of medicament into three separate doses, although in further embodiment, the medicament may be divided into more than three doses. Such dose dividing can be envisaged by providing an appropriate number of blocking elements, and proportional length of plunger rod 12.

In use of the device of FIGS. 15-20, the device 10 starts in the initial position shown in FIG. 15. Here, the plunger rod 12 is in its start position and none of the medicament 19 has been expelled from within the chamber 15. Also in this initial position, at least one of the blocking elements 21a, 21b may be secured to the finger flange 13 by the locking formations 26, 27, and is unable to be slid off the finger flange 13 since the first region 30 of the plunger rod 12 is located in the aperture 23 and so cannot pass through the opening 24. In this embodiment, similar locking formations as described above (e.g. tongue and groove) may be provided on adjacent surfaces of the first and second blocking elements 21a, 21b, such that the second blocking element 21b is secured to the finger flange 13, and the first blocking element 21a is secured to the second blocking element 21b.

The user may be a patient them self if self-administered, or a healthcare professional or other user if the medicament is being administered to the patient by a third party. The user presses the end surface 20 with their thumb, with their index and middle fingers on the finger flange 13, to move the plunger rod 12 axially in a distal direction D. This pushes the piston 18 distally within the chamber 15 and expels the medicament 19 out of the outlet 16 and through the medicament passage of the atomiser 17 to atomise the medicament for inhalation by a patient. The user continues to move the plunger rod 12 distally over this first range of motion until the distal face of the end surface 20 abuts the proximal face of the first blocking element 21a, as shown in FIG. 16, and the plunger rod 12 therefore cannot move any further in the distal direction.

At this point, the plunger rod 12 is in a first intermediate position and a first dose of the medicament 19 has been delivered to a patient. Also at this point, first region 30 of the plunger rod 12 has moved distally such that the second region 31 of the plunger rod 12 is positioned within the aperture 23 of the first blocking element 21a. Since the dimension d4 of the second region 31 of the plunger rod 12 and the dimension d2 of the opening 24 in the first blocking element 21a are configured as described above, namely that the second region 31 of the plunger rod 12 can pass through the opening 24, the first blocking element 21a is no longer locked to the plunger rod 12.

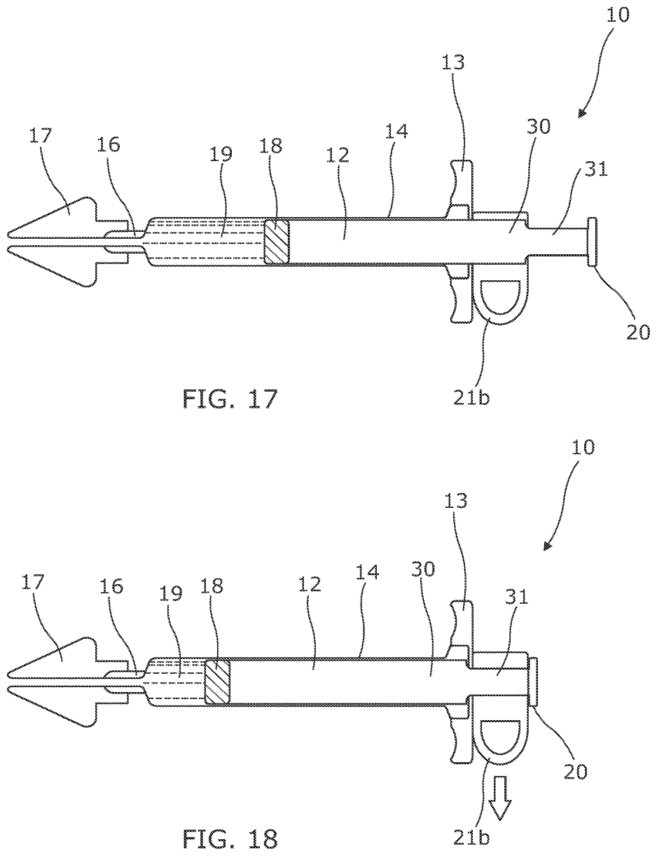

The user then slides the first blocking element 21a off the plunger rod 12 as shown by the arrow in FIG. 16, helped by gripping the grip region 25, so that the device 10 is in the configuration as shown in FIG. 17. That is, the plunger rod 12 is in a first intermediate position having been moved over a first range of motion but there remains medicament 19 within the chamber 15 for subsequent doses to be delivered. Since the first blocking element 21a has been removed, the plunger rod 12 is no longer prevented from moving further axially in the distal direction. The user then presses the end surface 20 again to move the plunger rod 12 axially in a distal direction D which pushes the piston 18 distally within the chamber 15 and expels further medicament 19 out of the outlet 16 and through the medicament passage of the atomiser 17 to atomise the medicament for inhalation by a patient. The user continues to move the plunger rod 12 distally over this second range of motion until the distal face of the end surface 20 abuts the proximal face of the second dose divider 21b and therefore cannot move any further in the distal direction.

At this point, the plunger rod 12 is in a second intermediate position, shown in FIG. 18, and a second dose of the medicament 19 has been delivered to a patient. Also at this point, first region 30 of the plunger rod 12 has moved distally such that the second region 31 of the plunger rod 12 is positioned within the aperture 23 of the second blocking element 21b. Since the dimension d4 of the second region 31 of the plunger rod 12 and the dimension d2 of the opening 24 in the second blocking element 21b are configured as described above, namely that the second region 31 of the plunger rod 12 can pass through the opening 24, the second blocking element 21b is no longer locked to the plunger rod 12.

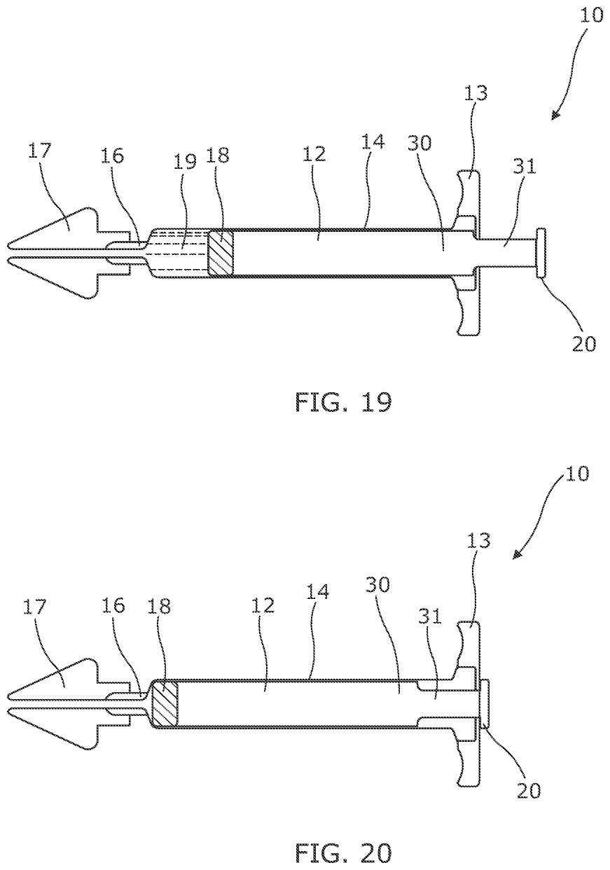

The user then slides the second blocking element 21b off the plunger rod 12 as shown by the arrow in FIG. 18, helped by gripping the grip region 25, so that the device 10 is in the configuration as shown in FIG. 19. That is, the plunger rod 12 is in a second intermediate position having been moved over the first and second ranges of motion but there remains medicament 19 within the chamber 15 for a subsequent dose to be delivered. Since the second blocking element 21b has been removed, the plunger rod 12 is no longer prevented from moving further axially in the distal direction. The user then presses the end surface 20 again to move the plunger rod 12 axially in a distal direction D which pushes the piston 18 distally within the chamber 15 and expels the remaining medicament 19 out of the outlet 16 and through the medicament passage of the atomiser 17 to atomise the medicament for inhalation by a patient. The user continues to move the plunger rod 12 distally over this third range of motion until either the distal face of the end surface 20 abuts the finger flange 13, or the piston 18 abuts the distal end of the chamber 15 and therefore cannot move any further in the distal direction. At this point, the plunger rod 12 is in an end position and the remaining medicament 19 has been expelled as a third and final dose delivered to the patient.

The device 10 described above provides a simple and effective way to ensure a user is able to divide the medicament delivery process into three separate doses, and the blocking elements 21a, 21b provides an effective way to ensure the medicament 19 is divided as intended and the user cannot accidentally deliver too much/all of the medicament in the first and second doses. Furthermore, since the blocking elements 21a, 21b are not removable from the plunger rod 12 until the plunger rod 12 has moved over the respective first and second ranges of motion, the blocking elements 21a, 21b cannot be tampered with and removed before the respective first and second doses of medicament 19 are delivered. This further helps towards ensuring the medicament is delivered in the intended dose volumes in each dose.

As with the embodiment described previously, lengths L2a, L2b of the first and second blocking elements 21a, 21b in an axial direction of the plunger rod 12, and the distance L1 corresponding to the distance between the proximal end of the first blocking element 21a and the distal face of the end surface 20 (not shown in FIG. 15 but as shown in FIG. 2), have a length dimension in the axial direction which can be selected such that the volume of medicament 19 delivered in each dose is equal. The ratio of volumes of first, second and third doses of medicament to be delivered can be controlled by controlling the first, second and third ranges of motion of the plunger rod 12 by selecting dimensions L2a, L2b of the respective first and second blocking elements 21a, 21b, as well as distance L1 of the system in the initial position. In some embodiments, these dimensions may be selected to deliver different volumes of medicament in each dose delivery.

Although the above-described embodiments comprise locking formations to secure the blocking element to the container 11 or adjacent blocking element 21a, 21b, it is envisaged that alternative embodiments may omit locking formations. It will be appreciated that the locking formations help prevent removal of the blocking element before initial use of the device 10 by sliding the blocking element 21, 21a, 21b proximally along the plunger rod 12 to the second region 31 and removing it. However, the dose dividing function of the blocking element 21 may still be achieved in the absence of such locking formations.

The above-described embodiments comprise one or more blocking elements 21 which may be a dose clip which may be removably attached to the plunger rod 12. As used herein, the term “dose clip” is intended to encompass components which may be substantially rigid components. This is, components which do not have moveable element(s), or element(s) which are required to be moved in order for the blocking element/dose clip 21 to be removed from the piston rod 12. This may help towards simplicity of design and manufacture, and ease of use. This may also help avoid unintentional/premature removal of the blocking element/dose clip 21 before a first dose of medicament has been expelled. That is, the blocking element/dose clip may be prevented from being removed from the device 10 until the plunger rod has been moved over a first range of motion as the blocking element/dose clip 21 cannot be manipulated, deflected or altered in shape by a user to render it releasable from the plunger rod 12.

Described herein are systems, devices, components, and methods that may be associated with delivering a respiratory syncytial virus (RSV) vaccine using an intranasal atomization delivery device.

As used herein, “RSV ΔNS2/Δ1313/11314L” refers to an RSV ΔNS2/Δ1313/11314L (NIH) or an RSV ΔNS2/Δ1313/11314L (Sanofi). Each of the ΔNS2/Δ1313/11314L (NIH) and the RSV ΔNS 2/Δ1313/11314L (Sanofi) comprise a live-attenuated RSV with (i) a 523 nucleotide (nt) deletion of the NS2 gene (ΔNS2), (ii) an amino acid deletion in the L protein, and (iii) a genetically stabilizing mutation in the L gene. The live-attenuated RSV of the RSV ΔNS2/Δ1313/11314L (Sanofi) also includes a nucleotide modification at position 14456 that represents a change from a thymine (T) to an adenine (A) in a non-coding region.

As used herein, “RSV ΔNS2/Δ1313/11314L vaccine” refers to an “RSV ΔNS2/Δ1313/11314L (NIH) vaccine” or an “RSV ΔNS2/Δ1313/11314L (Sanofi) vaccine.” An RSV ΔNS2/Δ1313/11314L (NIH) vaccine comprises an effective amount of RSV ΔNS2/A1313/11314L (NIH). An RSV ΔNS2/Δ1313/11314L (Sanofi) vaccine comprises an effective amount of RSV ΔNS2/Δ1313/11314L (Sanofi).

### Exemplary Methods and Uses

In some embodiments, provided are methods of delivering a dose of an RSV vaccine using an intranasal atomization delivery device.

In some embodiments, the methods of administering a dose of an RSV vaccine that comprises a live attenuated RSV use an intranasal atomization delivery device. In some embodiments, provided are methods of administering a dose of an RSV vaccine using an intranasal atomization delivery device, the RSV vaccine comprising an effective amount of a live-attenuated RSV with (i) a 523 nucleotide (nt) deletion of the NS2 gene (ΔNS2), (ii) an amino acid deletion in the L protein, and (iii) a genetically stabilizing mutation in the L gene (RSVΔ NS2/Δ1313/11314L (NIH) vaccine) or RSVΔ NS2/Δ1313/11314L (Sanofi) vaccine that further comprises a nucleotide modification in a non-coding region that represents a change from a thymine (T) to an adenine (A). In some embodiments, a codon that encodes a serine at position 1313 of the L protein is deleted resulting in the deletion of the amino acid in the L protein (Δ1313). In some embodiments, an amino acid residue substitution of leucine for isoleucine at position 1314 results in a genetically stabilizing mutation in the L gene (11314L).

In some embodiments, provided are methods of administering a dose of an RSV vaccine using an intranasal atomization delivery device. In some embodiments, the paediatric subject may be 6 to 18 months of age.

In some embodiments, the RSV vaccine is delivered intranasally using the intranasal atomization delivery device to deliver about ½ dose to each nostril. In some embodiments, the RSV vaccine is delivered intranasally so that the whole dose is delivered to one nostril. In some embodiments, the RSV vaccine is delivered intranasally with ½ dose delivered to one nostril and the other ½ dose is delivered to the same nostril after the first ½ dose is absorbed. In some embodiments, the RSV vaccine is delivered intranasally using the intranasal atomization delivery device to deliver a dose in unequal amounts to one or both nostrils.

In some embodiments, the RSV vaccine is delivered intranasally using the intranasal atomization delivery device in a liquid formulation. In some embodiments, the RSV vaccine dose is delivered intranasally using the intranasal atomization delivery device in about 0.2 mL. In some embodiments, the 0.2 mL dose is delivered intranasally wherein about 0.1 mL is delivered to each nostril. In some embodiments, the RSV vaccine dose is delivered intranasally using the intranasal atomization delivery device in about 0.01 mL, 0.02 mL, 0.05 mL, 0.1 mL, 0.2 mL, 0.3 mL, 0.4 mL, 0.5 mL, 0.6 mL, 0.7 mL, 0.8 mL, 0.9 mL, 1.0 mL, 1.1 mL, 1.2 mL, 1.3 mL, 1.4 mL or 1.5 mL. As described above, the dose may be divided evenly between two nostrils, divided unevenly between two nostrils, or delivered all to one nostril in one or more deliveries.

In some embodiments, provided are methods of administering a first dose of an RSV vaccine using an intranasal atomization delivery device and administering a second dose of the RSV vaccine using an intranasal atomization delivery device.

In some embodiments, provided are methods of administering a first dose of an RSV vaccine using an intranasal atomization delivery device wherein the intranasal atomization delivery device comprises a spray nozzle to atomize the RSV vaccine and direct a spray plume toward a top of a nasal passageway into a nasal cavity. In some embodiments, provided are methods of administering a dose of an RSV vaccine using an intranasal atomization delivery device wherein the intranasal atomization delivery device comprises a spray nozzle to atomize the RSV vaccine and direct a spray plume toward a top of a nasal passageway into a nasal cavity. In some embodiments, the intranasal atomization delivery device comprises a barrel operably connected to the spray nozzle and a plunger movable within the barrel to advance the RSV vaccine through the spray nozzle. In some embodiments, the intranasal atomization delivery device further includes a dose divider for splitting the dose of the RSV vaccine into two or more deliveries. In some embodiments, the dose divider may divide the dose of the RSV vaccine in about half of a volume to be delivered to a subject. In some embodiments the dose divider is used to deliver ½ dose to each nostril of the subject.

In some embodiments, the intranasal atomization delivery device delivers an average droplet size Dᵥ₅₀ of 10-120 μm. In some embodiments, the intranasal atomization delivery device delivers an average droplet size Dᵥ₅₀ of 10-120 μm, about 30-110 μm, about 50-110 μm, about 70-110 μm, or about 80-110 μm. In some embodiments, the intranasal atomization delivery device delivers an average shot weight between about 95 mg to about 135 mg, between about 100 mg to about 130 mg, or between about 100 mg to about 130 mg or between about 105 mg to about 130. In some embodiments, the intranasal atomization delivery device delivers an average shot volume of about 85 μL to about 120 μL, about 90 μL to about 115 μL, or about 95 μL to about 115 μL.

In some embodiments, use of an intranasal atomization delivery device described herein for administering a dose of an RSV vaccine to a subject is provided wherein the RSV vaccine comprises an effective amount of a live-attenuated RSV. In some embodiments, the use includes a live-attenuated RSV with (i) a 523 nucleotide (nt) deletion of the NS2 gene (ΔNS2), (ii) an amino acid deletion in the L protein, and (iii) a genetically stabilizing mutation in the L gene (RSVΔ NS2/Δ1313/11314L) (NIH) vaccine or RSVΔ NS2/Δ1313/11314L (Sanofi) vaccine that further comprises a nucleotide modification in a non-coding region that represents a change from a thymine (T) to an adenine (A).

### Immunogenicity

Approximately seventy percent (70%) of vaccine recipients attained a 4-fold response in neutralizing antibody titer following the second vaccine dose in both low and high dose groups, compared to 61 and 47% following one vaccine dose in low and high dose RSV naïve participants. This increase in the percentage of participants attaining a 4-fold response following a second administration supports the use of a second vaccine administration in this population. The 70% attaining this fold rise post second vaccine administration aligns with the expected clinical efficacy goal of 70% for the candidate. After one or two vaccinations, 75% attained a 4-fold rise in serum neutralizing antibody titers. The percentage of RSV experienced participants (36% and 22% in the low and high dose groups respectively) also attaining a 4-fold response in neutralizing antibody titers post vaccination 1 suggests potential benefit for this sub-group as well. It must be noted that this comes from a modest sample of participants (n=20 of 97 vaccine recipients) at this point of interim analysis.

Taken together, these data strongly support the use of RSV ΔNS2/Δ1313/11314L (Sanofi) vaccine at an operative range study for the candidate including doses at 5.6 and 6.2 log PFU/dose.

### Vaccine Virus Shedding and Infectivity

Following each vaccine administration, the shedding of vaccine virus was considered in addition to the fold rise in neutralizing antibody titers or serum IgG as vaccine infectivity. The high level of vaccine infectivity (over 80% and 70% in RSV naïve following the first and second vaccine administration respectively) is supportive of a promising vaccine candidate associated with good vaccine take. When infectivity was considered after either vaccine administration, over 90% of participants had evidence of infection. In the small cohort of RSV experienced participants, relatively high infectivity (80% and 33.3% in low and high dose recipients following one vaccine administration and 60 and 50% following a second vaccine administration) was found, implicating promise for this group. In addition, the marked drop in the percentage of vaccine virus shedders in RSV naïve participants after the second vaccine administration (roughly 20%) compared to the first vaccine administration (over 70%) is as previously documented with other efficacious live-attenuated mucosal viral vaccines where subsequent ‘challenge’ in the form of a second vaccine dose is characterized by a marked reduction in vaccine virus shedding. Of note, the shedding data available for this cohort was from data at a single timepoint following each vaccination (seven days post vaccination). While this coincides with the point of peak viral shedding documented in other RSV live-attenuated vaccine (LAV) trials, it is likely that some shedders may have been missed. This limitation in the available shedding data makes the results obtained particularly encouraging.

Interim analysis results showed a promising safety, immunogenicity and infectivity profile of the RSV ΔNS2/Δ1313/11314L (Sanofi) candidate.

No safety concerns were identified after 1-and 2-dose administrations of either dose level of the investigational RSV ΔNS2/Δ1313/11314L (Sanofi) vaccine or by baseline serostatus.

The vaccine virus shedding, and immunogenicity conclusions based on the IgA serostatus at baseline show marked vaccine take demonstrated at both dose levels, and 70% of vaccine RSV-naïve recipients attained a 4-fold response in serum neutralizing antibody responses post the second vaccine administration for both dose levels in RSV-naïve participants.

### REFERENCE NUMERAL KEY

10 medicament delivery system
11 medicament container/syringe
12 plunger rod
13 finger flange
14 outer side wall of container
15 medicament chamber
16 outlet
17 atomiser
18 piston
19 medicament
20 end surface
21 blocking element/dose divider/dose clip
22 body of blocking element
23 aperture through blocking element
24 opening in blocking element
25 grip region of blocking element
26 locking formation on blocking element
27 locking formation on finger flange
28 outwardly tapering edges of rib 26
29 outwardly tapering edges of slot 27
30 first region of plunger rod
31 second region of plunger rod
40 apparatus comprises finger flange 13, blocking element 21 and plunger rod 12
41 syringe barrel
42 syringe lip
43 projections of finger flange

1. A medicament delivery system (10) comprising:
a syringe (11) comprising a chamber (15) for containing medicament and an outlet (16) at a distal end of the syringe through which medicament (19) can be expelled from the chamber;
a plunger rod (12) mounted within the syringe and slidable relative to the syringe to cause medicament to be expelled from the chamber when medicament is within the chamber, wherein the plunger rod comprises a first region (30) of a first outer configuration and a second region (31) of a second outer configuration that is different to the first outer configuration; and
a blocking element (21) configured to be arranged at a proximal end of the syringe and disposed around the plunger rod, and which permits the plunger rod to move over a first range of motion relative to the syringe to expel a first dose of medicament, and blocks the plunger rod from moving over a subsequent range of motion relative to the syringe to expel further medicament;
wherein the blocking element (21) is configured to be removable from the plunger rod; and
wherein the plunger rod is free to be moved over the subsequent range of motion relative to the syringe to expel further medicament once the blocking element is removed from the plunger rod.

2. A medicament delivery system (10) according to claim 1, wherein when the plunger rod (12) is located along the first range of motion relative to the syringe (11), the blocking element (21) permits the plunger rod to move over the first range of motion, and wherein the blocking element becomes removable from the plunger rod once the plunger rod has completed the first range of motion.

3. A medicament delivery system (10) according to claim 1 or claim 2, wherein the blocking element (21) is secured to the plunger rod (12) when the plunger rod is in a first position relative to the syringe (11), and wherein the blocking element is removable from the plunger rod when the plunger rod (12) is in a second position relative to the syringe.

4. A medicament delivery system (10) according to any one of the preceding claims, wherein the blocking element (21) is secured to the syringe (11) whilst the plunger rod (12) is located along the first range of motion and is removable from the syringe when the plunger rod has completed the first range of motion.

5. A medicament delivery system (10) according to any one of the preceding claims, wherein the blocking element (21) comprises a locking formation (26, 27) configured to fix the blocking element relative to the syringe (11) whilst the plunger rod (12) is moveable over the first range of motion.

6. A medicament delivery system (10) according to claim 5, wherein the locking formation (26, 27) comprises one of a rib or groove that is arranged to be in sliding engagement with the other of a groove or rib formed on the syringe.

7. A medicament delivery system according to any one of the preceding claims, comprising a finger flange (13) at the proximal end of the syringe, and optionally wherein the syringe comprises a lip (42) at the proximal end, and the finger flange (13) is connected to the lip of the syringe.

8. A medicament delivery system (10) according to any one of the preceding claims, wherein the blocking element (21) comprises an aperture (23) and an opening (24) on a side of the blocking element in communication with the aperture, and the plunger rod is slidable through the aperture in the blocking element.

9. A medicament delivery system (10) according to claim 8, wherein the first region cannot pass through the opening (24) in blocking element (21), preventing removal of the blocking element from the plunger rod, and
wherein the second region is able to pass through the opening in the blocking element, permitting removal of the blocking element from the plunger rod.

10. A medicament delivery system (10) according to any one of the preceding claims, wherein the first region (30) comprises a first outer dimension, and the second region (31) comprises a second outer dimension that is smaller than the first outer dimension.

11. A medicament delivery system (10) according to any one of the preceding claims, wherein the blocking element (21) includes a grip region (25) to facilitate removal of the blocking element (21) from at least one of the plunger rod or the syringe.

12. A medicament delivery system (10) according to any one of the preceding claims, wherein the plunger rod (12) and the blocking element (21) are configured such that substantially half of the medicament (19) in the chamber (15) is expelled as the plunger rod moves over the first range of motion, and substantially half of the medicament in the chamber is expelled as the plunger rod moves over a second range of motion.

13. A medicament delivery system (10) according to any one of the preceding claims, further comprising an atomiser (17) at the outlet (16) of the syringe to facilitate insertion of the atomiser and the outlet into an orifice of a patient into which the medicament is to be delivered.

14. A medicament delivery system (10) according to any one of the preceding claims, wherein medicament (19) is received within the chamber (15) of the syringe (11).

15. An apparatus comprising:
a finger flange (13),
a blocking element (21), and
a plunger rod (12) for use with a syringe (11), the plunger rod receivable within an aperture (23) in the blocking element, wherein the plunger rod comprises a first region (30) of a first outer configuration and a second region (31) of a second outer configuration that is different to the first outer configuration,
wherein the apparatus is attachable to the syringe such that the plunger rod is slidable within and relative to the syringe, and such that the blocking element permits the plunger rod to move over a first range of motion relative to the syringe to expel a first dose of medicament, and blocks the plunger rod from moving over a subsequent range of motion relative to the syringe to expel a further dose of medicament.

16. An apparatus according to claim 15, wherein when the plunger rod (12) is located along the first range of motion relative to the syringe (11), the blocking element (21) permits the plunger rod to move over the first range of motion, and wherein the blocking element becomes removable from the plunger rod once the plunger rod has completed the first range of motion.

17. A method of operating a medicament delivery system (10) comprising a syringe (11) comprising a chamber (15) for containing medicament (16) and an outlet (16) at a distal end of the syringe, a plunger rod (12) mounted within the syringe and slidable relative to the syringe to cause medicament (19) to be expelled from the chamber when medicament is within the chamber, wherein the plunger rod comprises a first region (30) of a first outer configuration and a second region (31) of a second outer configuration that is different than the first outer configuration; and a blocking element (21) configured to be arranged at a proximal end of the syringe and disposed around the plunger rod, the method comprising:
moving the plunger rod over a first range of motion relative to the syringe to expel a first dose of medicament;
the blocking element blocking the plunger rod from moving over a subsequent range of motion relative to the syringe;
removing the blocking element from the plunger rod; and
moving the plunger rod over the subsequent range of motion relative to the syringe to expel a further dose of medicament.

18. A method according to claim 17, wherein when the plunger rod (12) is located along the first range of motion relative to the syringe (11), wherein the blocking element (21) permits the plunger rod to move over the first range of motion, and wherein the blocking element becomes removable from the plunger rod once the plunger rod has completed the first range of motion.

19. A method of operating a medicament delivery system (10), the method comprising:
moving a plunger rod (12) over a first range of motion relative to a syringe (11) to expel a first dose of medicament;
blocking the plunger rod, via a blocking element (21), from moving over a subsequent range of motion relative to the syringe;
removing the blocking element from the plunger rod; and
moving the plunger rod over the subsequent range of motion relative to the syringe to expel a further dose of medicament.

20. A method according to claim 19, wherein when the plunger rod (12) is located along the first range of motion relative to the syringe (11), wherein the blocking element (21) is configured to permit the plunger rod to move over the first range of motion, and wherein the blocking element becomes removable from the plunger rod once the plunger rod has completed the first range of motion.
